(19) United States
(12) Patent Application Publication
(10) Pub. No.: US 2010/0028446 A1
(43) Pub. Date: Feb. 4, 2010

(54) TRANSPORT OF DRUGS VIA THE BLOOD-BRAIN BARRIER BY MEANS OF APOLIPOPROTEINS

(76) Inventors: Jörg Kreuter, Bad Homburg (DE); Klaus Langer, Schöneck (DE); Sebastian Dreis, Frankfurt (DE); Telli Hekmatara, Frankfurt (DE)

Correspondence Address: D. PETER HOCHBERG CO. L.P.A., 1940 EAST 6TH STREET, CLEVELAND, OH, 44114, US

(21) Appl. No.: 12/449,408

(22) PCT Filed: Feb. 1, 2008
(86) PCT No.: PCT/EP2008/000822

§ 371 Date: Aug. 6, 2009

(30) Foreign Application Priority Data
Feb. 10, 2007 (DE) 10 2007 006 663.7

(51) Int. Cl. A61K 9/14; A61K 38/16; A61K 38/43; A61K 39/395

(52) U.S. Cl. 424/489; 514/12; 424/94.1; 424/130.1

(57) ABSTRACT

Combination preparations comprising at least one apolipoprotein as one component, and a medicinal agent to be transported via the blood-brain barrier to the central nervous system as a further component. The components are administered simultaneously, separately or sequentially. A method for administering a medicinal agent to the central nervous system is also provided.

### CROSS-REFERENCE TO RELATED APPLICATIONS

This application is a National Stage application of International Application No. PCT/EP2008/000822, filed on Feb. 1, 2008, which claims priority of German application number 10 2007 006 663.7, filed on Feb. 10, 2007, both of which are incorporated herein by reference in their entireties.

### BACKGROUND OF THE INVENTION

1. Field of the Invention

The present invention relates to combination preparations by which medicinal agents can be transported across the blood-brain barrier into the central nervous system. More particularly, the present invention relates to combination preparations that comprise at least one apolipoprotein as a component, and a medicinal agent as a further component. The present invention also relates to methods of administering a medicinal agent across the blood-brain barrier to the central nervous system by simultaneous, separate or sequential administration of the components of a combination preparation which comprises at least one apolipoprotein as one component and a medicinal agent as a further component.

2. Description of the Prior Art

The blood-brain barrier is a physiological barrier between the blood stream and the central nervous system, by means of which the milieu conditions of the central nervous system are kept separate from the conditions of the blood stream. Apolar substances are capable of crossing the blood-brain barrier, but polar or hydrophilic substances with a molecular weight higher than that of urea cannot enter the central nervous system via the interstitial spaces between the endothelial cells of the vascular wall but need to enter the central nervous system via the transport systems of the endothelial cells.

The blood-brain barrier effectively protects the brain from toxic substances which circulate in the blood stream. However, the blood-brain barrier also prevents many medicinal agents from entering the central nervous system which are suitable for treating diseases of the brain and the central nervous system such as Alzheimer's dementia, Parkinson's disease, epilepsy, schizophrenia, Huntington's chorea, bacterial and viral infections, as well as cancer. Most of these medicinal agents are hydrophilic to an extent that the blood-brain barrier prevents them from entering the central nervous system.

It is estimated that more than 90% of the cerebrally active medicinal agents are only insufficiently capable of crossing the blood-brain barrier. Therefore, systemic administration of a large number of medicinal agents that are meant to take effect in the central nervous system is not possible by peroral administration or by injection into the blood stream. To achieve an effect on the central nervous system with these active agents, it is necessary to apply alternative methods of administration. Usually, these alternative administration methods are invasive methods, for example the direct injection of the medicinal agent into the brain or opening the blood-brain barrier by means of a hyperosmolar solution.

For alternative, but non-invasive administration methods, carrier systems, for example liposomes or complex modified nanoparticles, are used wherein an active agent that is to be administered is incorporated, or to which the active agent is bound or attached. However, the production of such carrier systems involves great effort. In addition, from the medical point of view the non-natural components of these carrier systems are considered a problem. Thus, it is known, for example, that nanoparticles of polyalkylcyanoacrylates which are coated with polysorbate 80 (TWEEN® 80) are capable of crossing the blood-brain barrier in order to transport hydrophilic medicinal agents to the brain and produce pharmacological effects in the brain. It is, however, a drawback that polysorbate 80 is not physiological and that the transport of the medicinal agent-loaded polyalkylcyanoacrylate nanoparticles across the blood-brain barrier could be due to a toxic effect of Polysorbat 80. In addition, it is being discussed whether polyacrylates are initiators of autoimmune diseases.

In the printed publication EP 1 392 255 A1, active agent-loaded nanoparticles are described that are based on a hydrophilic protein or on a combination of hydrophilic proteins and to which apolipoprotein E is coupled covalently or via the avidin/biotin system. Using such nanoparticles, Dalargin was successfully transported across the blood-brain barrier.

Since, from the medical point of view, complex active-agent-loaded nanoparticles are not entirely without problems and as their production involves great effort, they have not yet become established in the treatment of diseases of the brain or central nervous system. There is a need for pharmaceutical preparations that can be produced much more easily and at a lower cost, for the administration of hydrophilic medicinal agents via the blood-brain barrier to the central nervous system.

### SUMMARY OF THE PRESENT INVENTION

It was therefore the object of the present invention to provide pharmaceutical preparations that are harmless from a medical point of view, can be produced more easily and at lower cost, and by means of which hydrophilic medicinal agents can enter the central nervous system via the blood-brain barrier so that they can produce a therapeutical effect in the central nervous system.

On solving this task, it was, surprisingly, found that a hydrophilic medicinal agent need not be bound to apolipoprotein-loaded nanoparticles in order to be able to be transported across the blood-brain barrier into the central nervous system. It was found that hydrophilic medicinal agents enter the central nervous system also if they are administered in combination, that is, simultaneously, separately or even sequentially, with an apolipoprotein by means of intravenous injection.

The present invention thus refers to combination preparations for administering a medicinal active agent to the central nervous system, comprising at least one apolipoprotein as component A and a medicinal agent as component B, for simultaneous, separate or sequential intravenous injection.

The invention comprises embodiments of the combination preparation wherein a mixture of apolipoprotein with medicinal agent is present in one common preparation.

The invention also encompasses embodiments of the combination preparation wherein apolipoprotein and medicinal agent are present in separate preparations, the preparation contains at least one apolipoprotein being free of medicinal agent to be administered, and the preparation contains the medicinal agent being free of apolipoproteins.

This embodiment enables the separate, even sequential administration of apolipoprotein and active agent, preferably by initially injecting the apolipoprotein-containing preparation, followed by administration of the medicinal agent-containing preparation.

The invention also encompasses methods wherein a medicinal agent is administered to the central nervous system by simultaneous, separate or sequential injection of at least one apolipoprotein.

### BRIEF DESCRIPTION OF THE DRAWINGS

FIG. 1 shows a graphic representation of the experimental results which illustrates a loperamide-mediated analgesic effect achieved by administration of apolipoprotein-loperamide solutions.

FIG. 2 shows a graphic representation of the experimental results, illustrating a loperamide-mediated analgesic effect after the sequential injection of apolipoprotein solution and loperamide solution.

### DETAILED DESCRIPTION OF THE PRESENT INVENTION

The terms “apolipoprotein” or “apoprotein” refer to proteins which are associated with the phospholipid monolayer of lipoproteins, including, but not limited to, apolipoprotein A (Apo A9), apolipoprotein B (Apo B), apolipoprotein D (Apo D), apolipoprotein E (Apo E) and all their isoforms.

The term “Apo A” means one or more of the isoforms of apo-lipoprotein A, including, but not limited to, Apo A-1.

The term “Apo B” means one or more of the isoforms of apo-lipoprotein B, including, but not limited to, Apo B-48 and Apo B-100.

The term “Apo E” means one or more of the isoforms of apo-lipoprotein E, including, but not limited to, Apo E-2, Apo E-3 and Apo E-4.

The term “medicinal agent” relates to pharmaceutical active agents which, when appropriately dosed, benefit human beings by serving to prevent, cure, alleviate or recognise diseases. The term medicinal agent comprises therapeutically beneficial amino acids, peptides, proteins, nucleic acids, including, but not limited to, oligonucleotides, polynucleotides, genes and the like, carbohydrates and lipids. The medicinal agents for the present invention also comprise cytostatic agents, neurotrophic factors, growth factors, pituitary hormones, hypothalamic hormones, enzymes, antibodies, neurotransmitters, neuromodulators, antibiotics, antiviral agents, antimycotics, chemotherapeutic agents, analgesics, psychopharmacologic agents, nootropics, anti-epileptic agents, sedatives and the like. The term “medicinal agents” encompasses both the medicinal agents which are active as such, as well as the “prodrugs” and precursors thereof, which can be activated when the active agent has reached the targeted tissue.

The term “pharmaceutically acceptable excipients” relates to chemical compositions or compounds with which a medicinal agent may be combined to enable production of a pharmaceutical form that is suitable for administration to humans. Pharmaceutically acceptable excipients comprise, without being limited thereto, carriers, tensides, inert diluents, granulating agents, disintegrants, binding agents, glidants, sweeteners, flavouring substances, colourants, preserving agents, physiologically acceptable compositions such as gelatine, aqueous carriers and solvents, oily carriers and solvents, suspending agents, dispersing agents or humectants, emulsifiers, demulsifiers, buffers, salts, thickening agents, fillers, antioxidants, stabilisers, polymeric or hydrophobic materials.

The combination preparations according to the present invention comprise at least one apolipoprotein (component A), and a medicinal agent (component B) which is to be administered to the central nervous system via the blood-brain barrier. The various embodiments of the inventive combination preparations enable simultaneous, separate or sequential administration of the two components.

Component A may be an apolipoprotein or a mixture of different apolipoproteins. Preferably, the apolipoproteins Apo A, Apo B and Apo E are being used, more preferably the apolipoproteins Apo A-1, Apo B-100 and Apo E-3, or mixtures of two or three of these isoforms.

Preferred medicinal agents (component B) for the combination preparation according to the invention are selected from the group consisting of nucleic acids, oligonucleotides, polynucleotides, genes, amino acids, peptides, proteins, pituitary hormones, hypothalamic hormones, neurotrophic factors, growth factors, antibodies, enzymes, carbohydrates, lipids, antiviral substances, antibiotics, antimycotics, cytostatics, analgesics, nootropics, antiepileptics, sedatives, psychopharmacologic agents, this list being by no means complete. With particular preference, the active agent is selected from the group consisting of dalargin, loperamide, tubocurarine, daunorubicin, and doxorubicin.

In one embodiment, component A and component B are present in one common preparation, which may additionally contain pharmaceutically acceptable excipients. This common preparation may be a solution, preferably an isotonic sodium chloride solution wherein both apolipoprotein and the active substance are dissolved.

It is, however, also possible for one of the two components or for both components to be present in the common preparation in the form of nanoparticulate formulations. This means that component A may be present in the form of medicinal agent-free nanoparticles to which apolipoprotein is coupled, and/or component B may be present in the form of medicinal agent-containing nanoparticles to which no apolipoprotein is coupled.

In another embodiment of the combination preparation according to the invention, components A and B are present in separate preparations, so that the two components can be administered separately from each other, simultaneously or in a sequential manner.

In this embodiment, too, components A and/or components B may be present in the form of solutions, preferably isotonic sodium chloride solutions, or in the form of nanoparticulate formulations in which the apolipoprotein-loaded nanoparticles are free of medicinal agent and the medicinal agent-containing nanoparticles are free of apolipoproteins.

Both the common preparation for the components A and B, and the separate preparations for the components A and B may, in addition to the respective components, contain pharmacologically acceptable excipients.

The present invention also relates to a method by means of which medicinal agents, particularly hydrophilic medicinal agents, can be administered to the central nervous system via the blood-brain barrier. This method for central nervous administration of a medicinal agent comprises the simultaneous, separate or sequential administration of the components of the combination preparation according to the present invention which comprises as component A at least one apolipoprotein, and as component B the medicinal agent to be administered.

To administer the combination preparation, that is, the components of the combination preparation, it is preferred to use the intravenous injection method.

The time delay in the sequential administration of the components A and B, which are present in separate preparations, in the case of which the apolipoprotein-containing preparation is administered first and the medicinal agent-containing preparation later, may amount to between 10 minutes and 24 hours. Preferably, the medicinal agent-containing preparation is administered within 480 minutes after the administration of the apolipoprotein-containing preparation. More preferably, the time lag amounts to between 15 minutes and 180 minutes. Still more preferably, the time lag amounts to between 30 minutes and 120 minutes, and most preferably between 60 minutes and 90 minutes.

### EXAMPLES

### Example 1

### Preparation of an Apolipoprotein E-3/Loperamide Solution

To prepare an apolipoprotein E-3/loperamide solution, 800 μg of lyophilised apolipoprotein E-3 were dissolved on a vortex shaker in 3.72 ml of a sterile isotonic NaCl solution. Subsequently, 280 μl of a loperamide primary solution (10 mg/ml) were added. The concentration of Apo E-3 in the resultant solution was 200 μg/ml and the concentration of loperamide in the resultant solution was 0.7 mg/ml.

### Example 2

### Preparation of an Apolipoprotein A-1/Loperamide Solution

To prepare an apolipoprotein A-1/loperamide solution, 800 μg of purified apolipoprotein A-1 was filled up with sterile isotonic NaCl solution to 3.72 ml.

Subsequently, 280 μl of a loperamide primary solution (10 mg/ml) were added and mixed with the vortex shaker. The concentration of Apo A1 in the resultant solution was 200 μg/ml and the concentration of loperamide was 0.7 mg/ml.

### Example 3

### Preparation of an Apolipoprotein B-100/Loperamide Solution

To prepare an apolipoprotein B-100/loperamide solution, 800 μg purified apolipoprotein B-100 were filled up with sterile isotonic NaCl solution to 3.72 ml.

Then, 280 μl of a loperamide primary solution (10 mg/ml) were added and mixed with the vortex shaker. The concentration of Apo B-100 in the resultant solution was 200 μg/ml and the concentration of loperamide was 0.7 mg/ml.

### Example 4

### Carrying Out the Animal Experiments

The apolipoprotein/loperamide solutions prepared in accordance with Examples 1 to 3 were injected into the tail vein of 10 mice (ICR/CD1) each. The dosage is relative to loperamide and amounted to 7.0 mg/kg body weight. This corresponded to an injection volume of 10 μL solution per gram of body weight of the mouse.

The tail-flick test was used to measure the analgesic effect of loperamide on the animals. To this end, the tail of each mouse was placed in a special apparatus above an infrared lamp and subjected to a heat stimulus. As soon as the pain stimulus became too strong, the mouse withdrew its tail, which triggered a photosensor which recorded the response time. The time measured was the time which passed until a mouse withdrew its tail from the heat source. To prevent any injuries to the animals, the measurement was truncated automatically after 10 seconds if the mouse showed no response to the heat stimulus.

The animals were tested after different points in time (15, 30, 45, 60, 120 and 180 minutes) after injection of the apolipoprotein/loperamide solution. From the values obtained, the maximum possible effect (MPE) was calculated using the below formula:

$$M P E ( \% ) = \frac{post \text{-} drug latency - pre \text{-} drug latency}{cut \text{-} off time - pre \text{-} drug latency} \times 100 \%$$

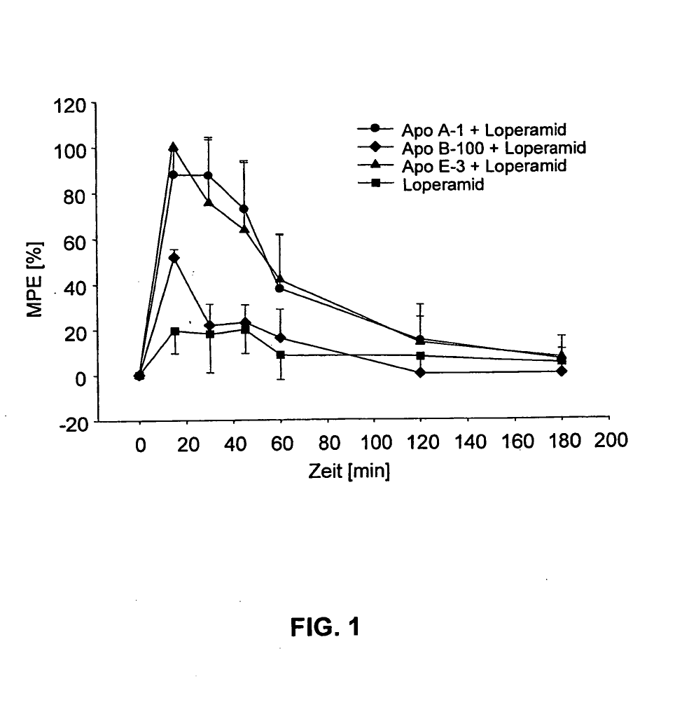

Using the apolipoprotein/loperamide solutions according to Examples 1 to 3 the results shown in FIG. 1 were achieved which demonstrate that with the apolipoprotein preparations the medicinal agent (loperamide) is transported across the blood-brain barrier whereas a loperamide solution without apolipoproteins did not lead to an analgesic effect.

### Example 5

### Preparation of an Apolipoprotein E-3 Solution

To prepare an apolipoprotein E-3 solution, 800 μg lyophilised apolipoprotein E-3 was dissolved on a vortex shaker in 4.0 ml of a sterile NaCl solution. The concentration of apolipoprotein E-3 in the resultant solution was 200 μg/ml.

### Example 6

### Preparation of a Loperamide Solution

To prepare a loperamide primary solution, 2.8 mg loperamide were dissolved in 280 μl ethanol 40.6% (v/v). To this primary solution were added 3.72 ml of a sterile isotonic NaCl solution. The concentration of loperamide in the resultant solution was 0.7 mg/ml.

### Example 7

### Performing the Animal Experiments with Sequential Administration of the Components

The solutions prepared according to Examples 5 and 6 were injected separately from each other into the tail vein of 10 mice (ICR/CD1) each. The apolipoprotein E-3 solution was applied first. The dosage is relative to apolipoprotein E-3 and was 2 mg/kg of body weight. This corresponded to an injection volume of 10 μl of solution per gram of body weight of the mouse.

At a time lag of 30 minutes, 120 minutes, 480 minutes or 24 hours after that first injection, the respective animal was subjected to an injection of the loperamide solution. The dosage is relative to loperamide and was 7 mg/kg. This corresponded to an injection volume of 10 μl solution per gram body weight of the mouse.

The analgesic effect of loperamide in the animals was measured and calculated in accordance with Example 4.

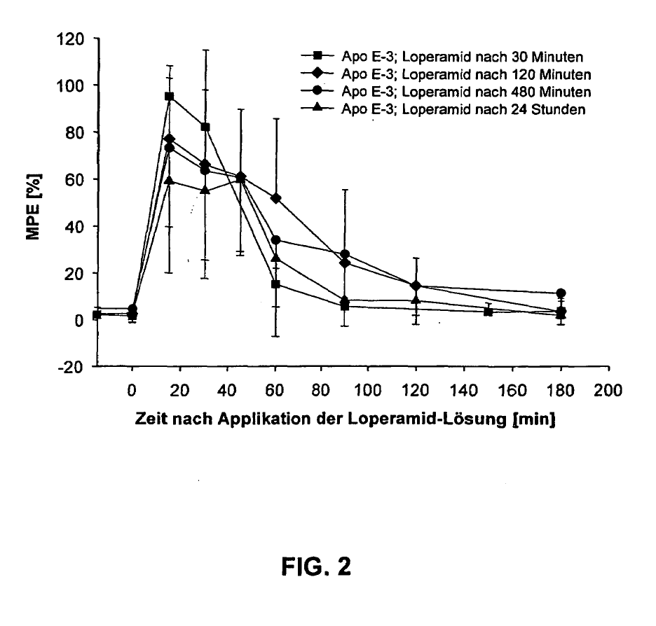

Using the apolipoprotein solution and the loperamide solution according to Examples 5 and 6, the results shown in FIG. 2 were achieved, which demonstrate that the medicinal agent (loperamide) is transported across the blood-brain barrier after a sequential application of the two components.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

We claim:

1. A combination preparation for administering a medicinal agent to the central nervous system, comprising at least one apolipoprotein as component A, and a medicinal agent as component B, for simultaneous, separate, or sequential intravenous injection, wherein
component A and component B are contained in one common preparation, said preparation being present in the form of a solution; or wherein
component A and/or component B is/are present in the form of nanoparticulate formulations in one common preparation, provided that component B is not bound to apolipoprotein-loaded nanoparticles; or wherein
components component A and component B are contained in separate preparations.

2. The combination preparation according to claim 1, wherein the apolipoprotein is selected from the group consisting of apolipoprotein A, apolipoprotein B and apolipoprotein E.

3. The combination preparation according to claim 2, wherein the apolipoprotein is selected from the group consisting of apolipoprotein A-1, apolipoprotein B-100 and apolipoprotein E-3.

4. The combination preparation according to claim 1, wherein the medicinal agent is selected from the group consisting of nucleic acids, oligonucleotides, polynucleotides, genes, amino acids, peptides, proteins, pituitary hormones, hypothalamic hormones, neurotrophic factors, growth factors, antibodies, enzymes, carbohydrates, lipids, antiviral substances, antibiotics, antimycotics, cytostatics, analgesics, nootropics, antiepileptics, sedatives and psychopharmacologic agents.

5. The combination preparation according to claim 4, wherein the medicinal agent is selected from the group consisting of doxorubicin, daunorubicin, dalargin, tubocurarine and loperamide.

6. A method for administering a medicinal agent to the central nervous system, said method comprising the step of simultaneously, separately or sequentially administering the components of a combination preparation comprising at least one apolipoprotein as component A, and a pharmaceutical active agent as component B, wherein component A and component B are administered in one common preparation as a mixture, or are administered as separate preparations.

7. The method according to claim 6, further comprising the step of administering the components A and B as separate preparations in a sequential manner, with the apolipoprotein-containing preparation being administered first, followed by the administration of the preparation which contains the pharmaceutical active agent.

8. The method according to claim 7, further comprising the step of administering components A and B by intravenous injection.

9. A combination preparation for intravenous injection for treating diseases of the central nervous system, said combination preparation comprising at least one apolipoprotein as component A, and a medicinal agent as component B, wherein component A and component B are present in one common preparation as a mixture, or wherein said components are contained in separate preparations.

10. The combination preparation according to claim 9, wherein said diseases to be treated are selected from the group consisting of Alzheimer's dementia, Parkinson's disease, epilepsy, schizophrenia, Huntington's chorea, bacterial and viral infections, and cancer.

11. The combination preparation according to claim 9, wherein the apolipoprotein is selected from the group consisting of apolipoprotein A, apolipoprotein B and apolipoprotein E.

12. The combination preparation according to claim 9, wherein the medicinal agent is selected from the group consisting of nucleic acids, oligonucleotides, polynucleotides, genes, amino acids, peptides, proteins, pituitary hormones, hypothalamic hormones, neurotrophic factors, growth factors, antibodies, enzymes, carbohydrates, lipids, antiviral substances, antibiotics, antimycotics, cytostatics, analgesics, nootropics, antiepileptics, sedatives and psychopharmacologic agents.

13. The combination preparation according to claim 12, wherein the medicinal agent is selected from the group consisting of doxorubicin, daunorubicin, dalargin, tubocurarine and loperamide.
